(19) United States
(12) Patent Application Publication
(10) Pub. No.: US 2021/0033624 A1
(43) Pub. Date: Feb. 4, 2021

(54) Online Measurement of Titer in Antibody Production

(71) Applicants: Guiyang Li, Thousand Oaks, CA (US); Tiansheng LI, Newbury Park, CA (US)

(72) Inventors: Guiyang Li, Thousand Oaks, CA (US); Tiansheng LI, Newbury Park, CA (US)

(21) Appl. No.: 16/839,019

(22) Filed: Apr. 2, 2020

Related U.S. Application Data
(60) Provisional application No. 62/879,942, filed on Jul. 29, 2019.

(51) Int. Cl. G01N 33/68; G01N 30/74; G01N 30/80; G01N 30/20

(52) U.S. Cl. CPC G01N 33/6854; G01N 30/20; G01N 30/80; G01N 30/74

(57) ABSTRACT

The present invention relates to a device and method capable of rapid and automated online measurement of antibody titers in antibody production. The device incorporates a fully automated affinity-column based instrument.

### PRIORITY CLAIM AND REFERENCE TO RELATED APPLICATION

This application claims the benefit of priority under 35 U.S.C. § 119(e) of U.S. Ser. No. 62/879,942, filed Jul. 29, 2019, the entire content of which is incorporated herein by reference.

### FIELD OF THE INVENTION

The present invention generally relates to device and method capable of rapid and automated online measurement of antibody titers in monoclonal antibody production. The methods incorporate a fully automated affinity-column based instrument and automated sampling system.

### BACKGROUND OF THE INVENTION

Biopharmaceutical and pharmaceutical industries are continuing to look for opportunities to shorten the product development cycle. They strive for producing better products that are comparable in quantity, quality, and efficacy to their competitors' similar products. Understanding the manufacturing process and being able to monitor and control the real-time quality of the product are keys in reducing costs and providing high quality products to patients. In order to achieve this goal, it will require the implementation of Process Analytical Technology (PAT) so that real-time product quality control can possibly be implemented.

Immunoglobulin G (IgG) antibodies are one of the most common and the most important biopharmaceutical medicines. They circulate in the blood and other body fluids, defending against invading bacteria and viruses. Most of the current antibody drugs are produced in mammalian CHO (Chinese Hamster Ovary) cells in a bioreactor, and production generally takes two weeks. Analytical techniques for measuring the amount of antibody protein (or called titer) are used in research, process development, and all stages of biotherapeutic drug manufacturing. The current technology generally uses high performance liquid chromatography (HPLC) to capture and elute with Protein A (ProA) affinity column to detect the signal of the antibody protein. Although there are many limitations, it is still the main force and considered as “gold standard”. This is an off-line technique that requires the samples to be removed from the bioreactors. Not only is the procedure cumbersome, but it also has the danger of contaminating the bioreactor and causes the entire production batch to fail.

Measurement of titers by HPLC, the “gold standard” method, is rarely accessible at-line, making measurements at multiple stages of the bioprocess challenging and inconvenient. Automated process analytical technology (PAT) to measure titer could help on monitoring the effects and variations of critical process parameters in real-time.

### SUMMARY OF THE INVENTION

The present invention provides a device and a method that can automate online measurement of titers in monoclonal antibody production.

A preferred embodiment provides an antibody titer measurement device. The device includes a housing that have a motor controlled syringe connected with a buffer valve to select either neutral carrier buffer to enable binding of antibody to a Protein A chromatographic column or acidic buffer to elute antibody. The device also provides a temporary storage coil connected to the syringe and a sample valve, acting as a temporary reservoir to enable delivery of the liquid antibody samples to ProA column. The sample valve includes a plurality of external valve ports and a central connection port connecting to temporary storage coil. The Protein A chromatographic column is connected to the valves to capture antibody proteins, to wash out impurities, and then to release antibody proteins into an ultraviolet detector. The ultraviolet detector measures the ultraviolet signal of the eluted antibody. The waste liquid collector takes the waste liquid.

A preferred embodiment also provides a method for online measuring antibody titers in an antibody production. The first step provides moving a connection line on a buffer valve to a neutral buffer, and filling a syringe with the neutral buffer; moving the connection line on the buffer valve to a temporary storage coil, and moving the connection line of a sample valve to a Protein A chromatographic column, injecting the neutral buffer in the syringe into the temporary coil, flowing through the Protein A chromatographic column and an ultraviolet detector, and to a waste liquid collector. The next step provides moving the sample valve to a bioreactor, storing a certain volume of antibody sample from the bioreactor in the corresponding temporary storage coil; moving the connection line in the buffer valve to neutral buffer, and filling the syringe with neutral buffer; moving the connection line on the buffer valve to the temporary storage coil, and moving the connection line through the sample valve to the ProA chromatographic column, flowing the antibody sample in the temporary storage coil to the ProA column through the sample valve; moving the connection line on the buffer valve to an acidic buffer, and filling the syringe with the acidic buffer; moving connection line on the motor to the temporary storage coil, the acidic buffer in the syringe flows into the temporary storage coil, through the sample valve to the ProA chromatographic column and to wash out the antibody sample into an ultraviolet detector. The ultraviolet signals will be collected to calculate the concentration of antibody sample.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein.

FIG. 1. An exemplary UV curve of antibody measurement in a CHO cell bioreactor sample by HPLC.

FIG. 2. Preferred key functional units of the device for automated online measurement of antibody titers in bioproduction.

FIG. 3. Preferred fluidic connection tube 300 for the device of FIG. 2.

FIG. 4. Preferred motorized syringe and buffer valve 400 for the device of FIG. 2.

FIG. 5A-5B. Preferred embodiment of buffer valve for the device of FIG. 2; front view (FIG. 5A) and back view (FIG. 5B) of the buffer valve, respectively.

FIG. 6. Preferred temporary storage coil 500 for the device of FIG. 2.

FIG. 7. Preferred motorized sample valve 600 for the device of FIG. 2.

FIG. 8A-8B. Preferred embodiment of sample valve for the device of FIG. 2. Front view (FIG. 8A) and back view (FIG. 8B) of the sample valve, respectively.

FIG. 9. Preferred embodiment with a ProA affinity column 700 for the device of FIG. 2.

FIG. 10. Preferred embodiment with a UV flowcell 800 for the device of FIG. 2.

FIG. 11. Exemplary diagram of UV monitoring of the eluted CHO cell culture matrix and antibody from a blank sample, purified standard IgG and a bioreactor sample.

FIG. 12. Linear regression of average peak area and antibody loading of standard samples.

FIG. 13. Exemplary online titer measurement by a prototype device for the device of FIG. 2.

### DETAILED DESCRIPTION OF THE INVENTION

The present invention provides a compact device that can automate online measurement of titers in monoclonal antibody production. The device incorporates a fully automated affinity (or capture) column and automatic sampling system.

The invention provides online measurement of antibody titers that avoids the needs for the samples to be analyzed offline, eliminates manual sampling, and greatly improves the efficiency and cost of the production. The online multiplex instrument could monitor multiple bioreactors of antibody protein production in real time. The antibody protein can be automatically pulled from the bioreactors, purified online, and then measured by UV absorbance and calculated by Beer's law. The value obtained will depend on the path length of the UV detector.

The Beer-Lambert law states that:

A (absorbance)=εcl
Where ε=extinction coefficient, c=concentration in mol/L and l=optical path length

Therefore, if extinction coefficient is known, measurement of A gives the concentration directly. The extinction coefficient could be measured or calculated. (Ref. Gill, S. C. and von Hippel, P. H. (1989) Calculation of protein extinction coefficients from amino acid sequence data. Analyt. Biochem. 182, 319-326.)

The device is small in size, and it provides sampling directly from the reactor, and automating the capture and elution of antibody proteins. The present invention allows automatic measurement of antibody protein concentration in multiple bioreactors through a motorized syringe, a motorized buffer valve, a temporary storage coil, a motorized multiposition sample valve, a Protein A (ProA) affinity column and a UV flowcell detector. The invention integrates automatic sampling system and automatic measurement.

The preferred embodiments include a temporary storage coil as temporary storage, enabling the purpose of withdrawing a sample from a plurality of bioreactors and delivering to a ProA column with a single syringe. The syringe of the present invention simultaneously provides the functions of extracting a sample, pushing the sample, and eluting the antibody protein from the column which led to multi-purpose functions.

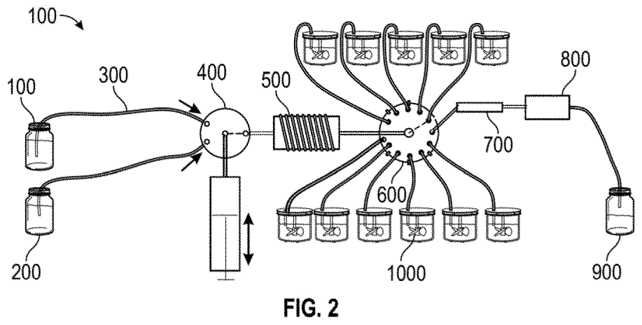

After the antibody is captured by ProA affinity column, it is eluted with acid buffer and the protein concentration is monitored by ultraviolet (UV) light. Generally, the motor-controlled syringe produces less pressure than HPLC, but the back pressure of the filler of the Protein A column can generally be controlled so as not to affect the use. The present invention improves the efficiency by avoiding the disadvantages of manual sampling and cumbersome design of the HPLC, which greatly simplifies the design of the circuit. The high pressure function of HPLC is critical for other columns, but is not required in this application. All processes are automated, and no manual operation is required, and the data result is direct output. Illustrated in FIG. 2 is an exemplary overview of the device with various preferred components of the present invention. The device comprises main functional units, including neutral buffer vessel 100, acidic buffer vessel 200, fluidic connection tube 300, motor controlled syringe 400, temporary storage coil 500, multi-position sample valve 600, ProA (Protein A) column 700, UV detector 800, waste bottle 900 and bioreactors 1000. As shown in FIG. 2, neutral buffer vessel 100 provides liquid for system operation under neutral conditions. Acidic buffer vessel 200 allows the antibody protein to be eluted and the signal is detected by ultraviolet detection under such conditions.

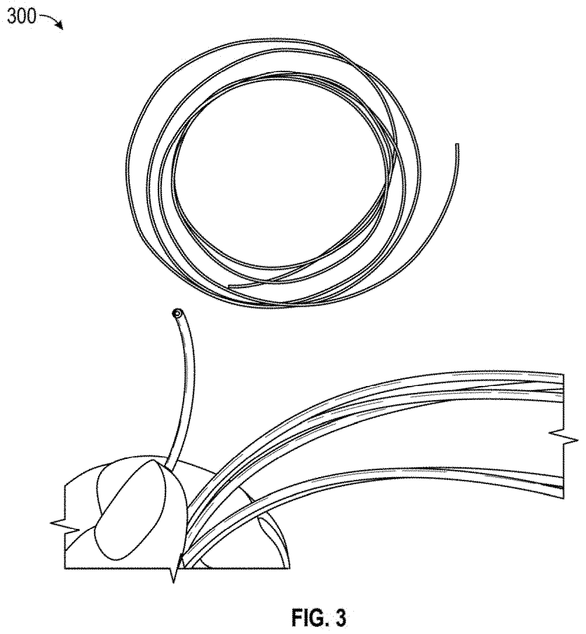

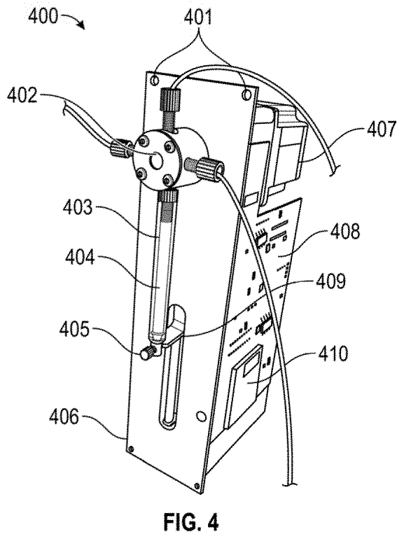

One preferred embodiment is the connection tube 300, as shown in FIG. 3. A Teflon tube with hollow channels in the middle provides connections of the integrated system. It connects the fluidic system and enables the flow of the liquids in the integrated system. In one preferred embodiment of the present disclosure, the motorized syringe is connected with the buffer valve 400 with the layout as shown in FIG. 4. The layout of the motorized syringe and the buffer valve 400 provide mounting holes 401, buffer valve 402, syringe 403, syringe plunger 404, plunger lock screw 405, front panel 406 syringe drive motor 407, electronic board 408, carriage 409, and valve drive motor 410.

The front plate 406 has mounting holes 401 on the top, which are designed to be fixed in the metal frames for integration purpose. The syringe 403 could be screwed into the buffer valve 402 with a bottom mount. The plunger 404 has a lock screw on the bottom 405 which is mounted on the carriage 409. The buffer valve's connection ports and the carriage could be controlled by separate drive motors 407 and 410. The electronic circuit board is used to digitally control the motors by commands programmed by an integration software.

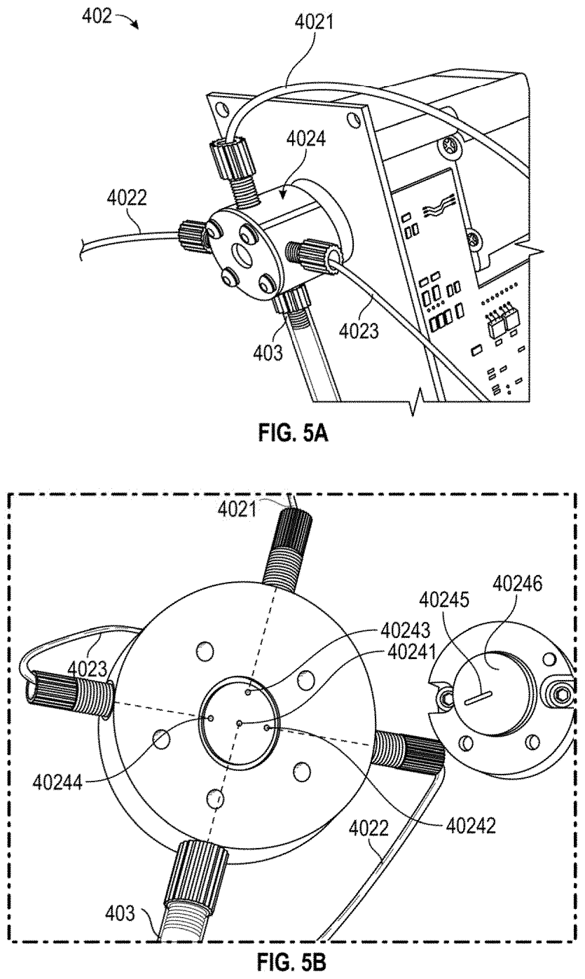

In one embodiment of the present disclosure, the buffer valve 402 is described in FIG. 5. The buffer valve in FIG. 5A has the syringe mount on the bottom, two tubing ports on the top for neutral buffer 4021 and acidic buffer 4022, and one port 4023 on the right to connect to temporary storage coil. Its rotatable plate 40246 has a straight groove 40245 (shown in FIG. 5B). It enables one of the three ports to connect with the bottom syringe 403.

The syringe could be connected with one of the three ports, neutral buffer 4021, acidic buffer 4022 and the temporary storage coil 4023 (FIG. 5 A). After the inner layout in valve head from the motor shaft comprises four openings, as shown in FIG. 5B. The four openings connect with the fluidic connection tubes. The middle opening 40241 connect with the syringe as illustrated by the dotted line. The openings 40244, 40243 and 40242 connect with connection tubes 4023 (to storage coil 300), 4021 (to acidic buffer 200) and 4022 (to neutral buffer) respectively. It has a rotatable plate 40246 in the middle to seal the system and provide connections through the straight groove 40245. The straight groove could be rotated and enable the connection of syringe 403 with one of the three ports, the neural buffer 200, the acidic buffer 100 or the temporary storage coil 300.

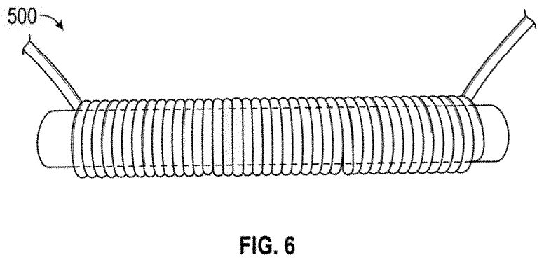

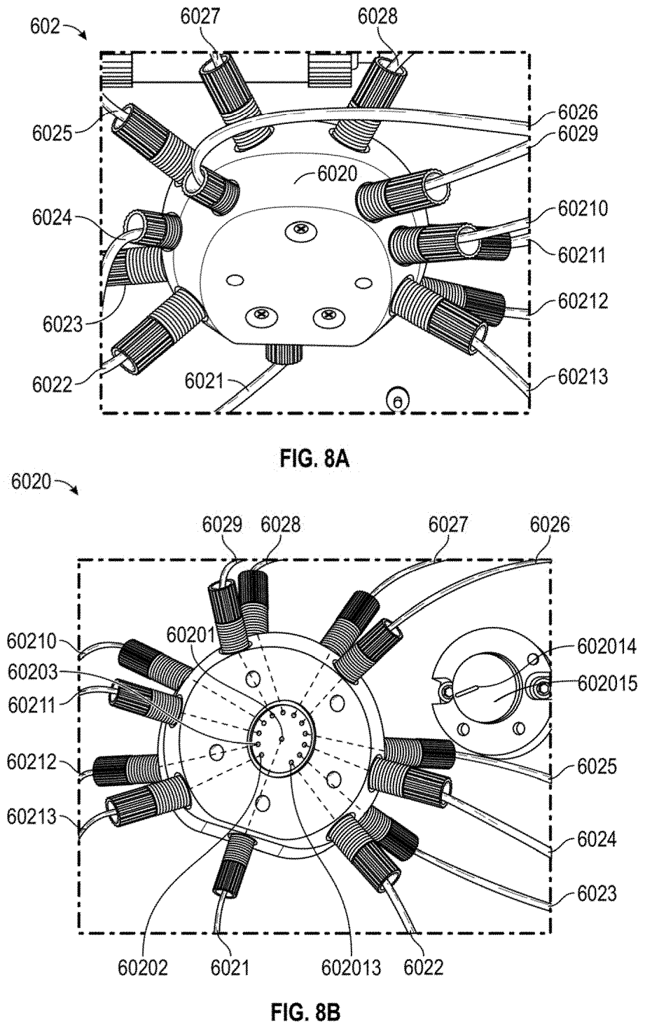

In one embodiment of the present disclosure, the temporary storage coil 500 is the long connection tubes coiled around a plastic cylinder with opening in the middle (FIG. 6). A long and thin connection tube is preferred to minimize the dilution of the buffer with our bioreactor samples. Temporary storage coil 500 provides temporary storage of bioreactor samples. The long connection tube is used to temporarily store the liquid bioreactor sample from one port and deliver it to ProA column port 6028 (FIG. 8) with minimized dilution by the buffer.

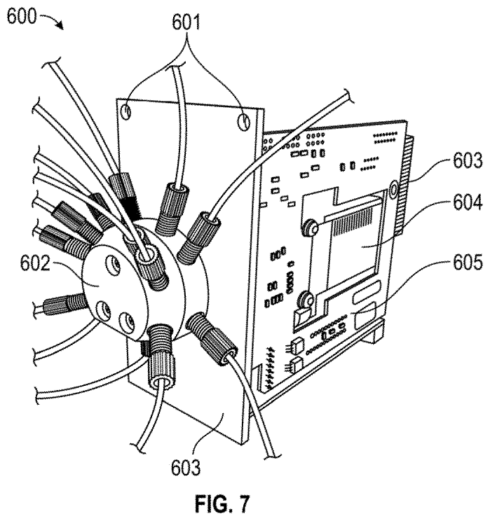

In one embodiment of the present disclosure, the layout of the multiposition sample valve 600 is as shown in FIG. 7. The front plate 603 has mounting holes 601 on the top, which are designed to be fixed to the frames for integration purpose. The connection of the ports on the valve head 602 could be controlled by the valve motor 604. The electronic circuit board 605 is used to digitally control the motors by commands programmed by the integration software. The board edge 603 provides communications with the integration software.

In one embodiment of the sample valve, the temporary storage coil tube 6021 could be connected with one of the twelve distribution ports (FIG. 8). Eleven ports could be used to connect with eleven bioreactors (ports 6022 through 6027 and 6029 through 60213). One port 6028 is used to connect with the ProA column 700. The front view of the valve is shown in FIG. 8A with the storage coil connection on the bottom port. The inner layout of the valve head from the motor shaft comprises of twelve peripheral openings and one central opening in FIG. 8B. A rotatable plate 602015 has a straight groove 602014 on it. One side of the straight groove in the middle constantly connects with the central opening 60201 which connects with the temporary storage coil 6021. The eleven bioreactor sample tubes and the ProA column port fluidly connect to the 12 peripheral openings. For example, the bioreactor sample tubes 60212, 60213 and 602013 have fluidic communications with the peripheral openings 60203, 60202 and 602013 respectively. The rotation of the plate 602015 will provide fluidic communications between bottom temporary storage coil 6021 with twelve peripheral ports.

The temporary storage coil is connected to the bottom port 6021 which connects to twelve ports on the valve head. The port 6028 connects to the ProA column and the rest eleven ports connect to bioreactors. The back of the valve head shows the temporary storage coil opening 60201 in the middle and the twelve peripheral openings for the peripheral ports. The rotatable plate 602015 has a straight groove 602014 to enable connection of storage coil with one of the twelve peripheral ports.

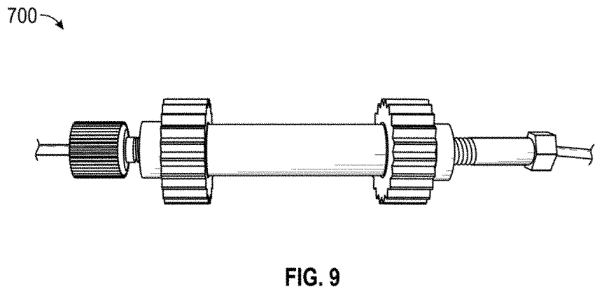

In one embodiment of the ProA affinity column 700, the ProA resin is packed in a cylindrical plastic tube with caps on both sides (FIG. 9). Each cap has an opening with a fitting for the connection tube. ProA affinity column 700 binds the antibody and enables the cleaning of cell culture matrix impurity.

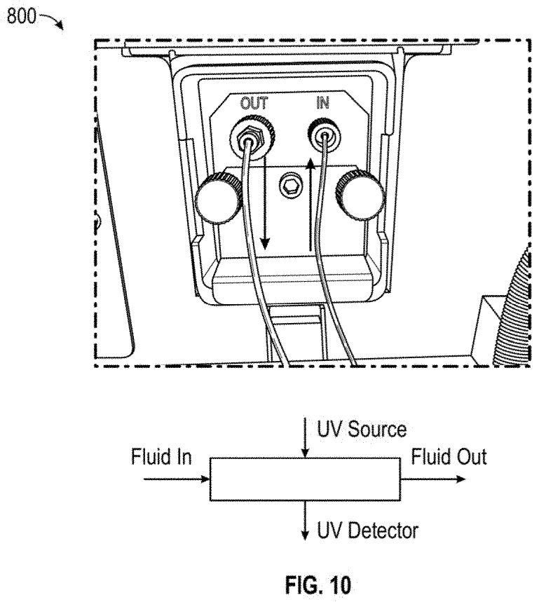

In one embodiment of the UV flowcell detector 800 (FIG. 10), the flow cell has metal openings to allow the liquid to move in for measurement and exit as waste to collection bottle 900. The detector measures the UV signal of the eluted antibody protein.

The invention also provides a method of online measurement of antibody titers in the antibody production.

The method involves multiple steps for the operation. The first step provides moving a connection line on a buffer valve to a neutral buffer, and filling a syringe with the neutral buffer; moving the connection line on the buffer valve to a temporary storage coil, and moving the connection line of a sample valve to a Protein A chromatographic column, injecting the neutral buffer in the syringe into the temporary coil, flowing through the Protein A chromatographic column and an ultraviolet detector, and to a waste liquid collector. The next step provides moving the sample valve to a bioreactor, storing a certain volume of antibody sample from the bioreactor in the corresponding temporary storage coil; moving the connection line in the buffer valve to neutral buffer, and filling the syringe with neutral buffer; moving the connection line on the buffer valve to the temporary storage coil, and moving the connection line through the sample valve to the ProA chromatographic column, flowing the antibody sample in the temporary storage coil to the ProA column through the sample valve; moving the connection line on the buffer valve to an acidic buffer, and filling the syringe with the acidic buffer; moving connection line on the motor to the temporary storage coil, the acidic buffer in the syringe flows into the temporary storage coil, through the sample valve to the ProA chromatographic column and to wash out the antibody sample into an ultraviolet detector. The ultraviolet signals will be collected to calculate the concentration of antibody sample.

The method is demonstrated by the following steps according to the device as shown in FIG. 2.
S1. The movable straight groove 40245 in the buffer valve head 402 moves to the neutral buffer 100, the syringe plunger 404 moves downward, and the syringe is filled with neutral buffer.
S2. The movable straight groove 40245 in the buffer valve head 402 moves to the temporary storage coil 500 and movable straight groove 602014 of the multi-positon sample valve 600 is moved to ProA 700, and the syringe plunger 404 is moved up to the top. The neutral buffer in the syringe is injected into the temporary storage coil 500, the ProA chromatographic column 700, the ultraviolet detector 800 and the waste liquid collector 900. The system was filled up with neutral buffer and the ProA column is conditioned.
S3. The movable straight groove 602014 of the multi-positon sample valve 600 moves to one of the bioreactor 1000. The syringe plunger 404 moves downward at a fixed precise distance. Certain volume of antibody samples from the bioreactor is stored in the corresponding temporary storage coil.
S4. The movable straight groove 40245 in the buffer valve head 402 moves to neutral buffer 100, the syringe plunger 404 moves downward, and the syringe is filled with neutral buffer.
S5. The movable straight groove 40245 in the buffer valve head 402 moves to the temporary storage coil 500 and movable straight groove 602014 of the multi-positon sample valve 600 is moved to ProA 700, and the syringe plunger 404 is moved up to the top. The antibody sample in the temporary coil 500 flows into the ProA column 700 through the sample valve 600. The antibody is captured by ProA column and other cell matrix impurities are washed out by the neutral buffer in the syringe.
S6. The movable straight groove 40245 in the buffer valve head 402 moves to the acidic buffer 200, the syringe plunger 404 moves downward, and the syringe is filled with the acid buffer.
S7. The movable straight groove 40245 in the buffer valve head 402 moves to the temporary storage coil 500, and the syringe plunger 404 is moved up to the top. The acidic buffer in the syringe flows into the temporary storage coil 500, through the sample valve 600 to the ProA chromatographic column 700, and the antibody proteins flow into the ultraviolet detector 800 to generate ultraviolet signals, which directly correspond to the concentration of antibody proteins.
S8. The antibody concentration in one of the bioreactors 1000 has been measured.
S9. Repeat the above steps, and movable straight groove 602014 of the multi-positon sample valve 600 moves to another bioreactor. In the same way, the concentration of the second bioreactor can be measured.
S10. Repeat step S9 with all the other bioreactor samples.

Bioreactors are complex ecosystems with host cells, nutrients, metabolites and biotherapy products, usually monoclonal antibodies. In the production of antibody drugs, it is necessary to optimize the production process and closely monitor the mixture. The amount of biotherapeutic protein produced is critical. The process of quantifying the product is also called titer measurement. During the development process, the composition of cell culture medium was optimized to maximize yield with as few impurities as possible. Understanding and control of the production process is essential for the regulatory approval of new biotherapeutic agents. A key aspect of the fermentation process in a bioreactor is the amount of antibody proteins (titer).

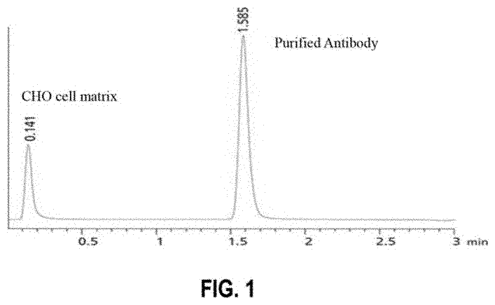

The combination of affinity capture and ultraviolet (UV) detection is the most commonly used measurement method so far. In the case of monoclonal antibodies, this usually means protein A (ProA) capture, although there are other options, such as for protein L for IgG3 antibodies, a special antibody which needs to act on the light chain protein L. Some therapeutic proteins require other affinity capture method, such as adding polyhistidine labels to enable nickel capture or Strep labels to enable Streptomyces antibiotic protein capture. Affinity capture is a simple two-step process. The sample was loaded onto the column and bound to the resin capture matrix by affinity. Then the target protein is eluted with an elution buffer after cleaning out the impurities. Protein A (ProA) affinity column is used the most to measure antibody production in bioreactors. Typically, the sample is loaded with a neutral pH mobile phase (usually a phosphate buffer) and then eluted with a low pH acidic mobile phase (such as citric acid or acetic acid solution) for ProA affinity column. Low pH can destroy the binding of antibody protein to ProA and elute it from the column. Accuracy is very important in titer measurement experiments, but other key factors are to be considered as well, such as speed, specificity, wide dynamic range and robustness. Many laboratories require measurements of a large number of samples, but even if this is not the case, information may need to be provided quickly so that decisions can be made as soon as possible. Many commercially available ProA affinity column can separate antibody drugs from the matrix in as little as 2 to 3 minutes using HPLC (as shown in FIG. 1). Affinity trapping is an ideal specificity to produce high purity protein products. Wide dynamic range ideally allows good sensitivity and relatively concentrated sample quantification without dilution.

The invention also provides alternative embodiments, as exemplified in the following features.

The multiposition sample valve and the buffer valve can be designed without rotating center piece. It can be configured in a similar function unit such as pinch valves by opening the required ones and closing other lines.

The motor controlled syringe can be changed to a peristaltic pump to provide power.

Column ProA can be exchanged for other types of capture columns. Such as protein L, metal ions and etc.

The sample valve could have more than 12 ports or it could connect to other multi-position sample valves through some of the 12 ports.

The elution conditions of the column will vary depending on the column.

The temporary storage temporary storage coil can be other temporary storage containers.

UV detectors can be exchanged for other measurement methods such as fluorescence detectors, mass spectrometry detectors, Charged Aerosol Detectors (CAD), light scattering, etc.

The antibodies can be full antibody or a fragment of antibodies or fusion proteins. The binding and elution mechanism will also work for other analytical purposes as long as the analyte could bind and elute with a solid stationary media.

### Example

A prototype device is made by assembling an integrated system following the device of FIG. 2. An UV flowcell from an Agilent HPLC model 1100 was utilized in the prototype device. The motorized syringe, buffer valve and the multi-position sample valves were digitally controlled by Carvo® software and the UV data were collected and analyzed by Chemstation® software.

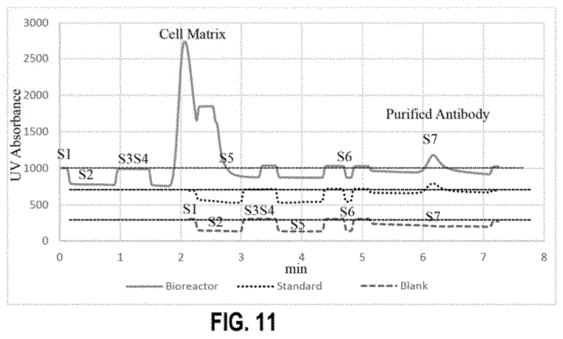

Blank, standard purified antibody and bioreactor samples were measured in the device. The relevant UV curves at 280 nm are shown in FIG. 11. The UV baselines of each sample are indicated with dotted lines and artificially shifted with offsets for better comparisons. The UV curves of the S1 through S6 of the blank or standard samples demonstrated that UV signal dropped as plateaus when the neutral buffer flow through the UV flow cell. When the flowrate remains constant, the baseline stabilizes at a lower level. When the buffer stops moving, the UV signal recovers to the original baseline level. The antibody signal should avoid these transition zones and was designed to place the UV signal of the antibody right in the middle of the dropped plateau of elution phase.

The operation steps S1 through S7 were labelled in the FIG. 11. It shows UV monitoring of the eluted CHO cell culture matrix and antibody from blank sample, purified standard IgG and a bioreactor sample. The corresponding steps of system operation, S1 through S7, were labelled above the UV curves. It should be noted that the S5 step is designed to clean the other impurities in the bioreactors when the antibody was bounded on the affinity column of ProA. In the bioreactor sample, the apparent large UV absorption right after 2 min peak are those impurities with strong UV absorbance substances. Two-stroke cleaning with 2 ml neutral buffer to clean the impurities were set up in the bioreactor sample. Whereas there is no absorption from impurities in the S5 of blank or standard samples as expected. The blank or standard sample is designed to have one stroke (1 ml) neural buffer washing S5 since no impurity is expected. Therefor the total operation time of the bioreactor sample is about 7 minutes and that of the standard or blank sample is about 5 minutes. In step S7, the antibody is eluted by acidic buffer. The bioreactor and the standard sample showed an absorption right after 6 min while blank sample doesn't show this peak.

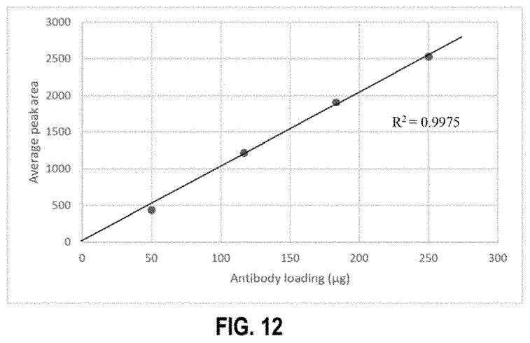

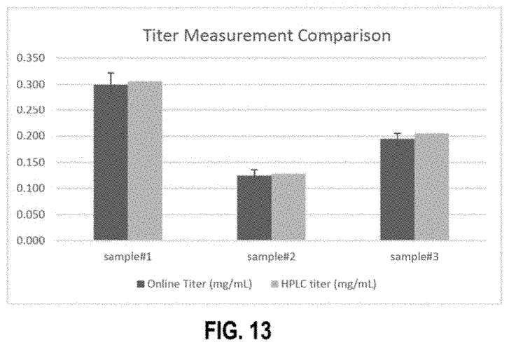

All together 4 standard samples were measured with different loadings on the affinity ProA column (Table 1). Each standard has different loadings on the affinity ProA column and was measured in three triplicates. In FIG. 12, linear regression of average peak area and antibody loading of standard samples. The relative standard deviations (RSD) are within 10% and the regression analysis generated a regression coefficient R² of 0.9975. This demonstrated a decent linearity and acceptable RSD. Three bioreactor samples were measured in triplicates (Table 2). The RSD were all within 10% and the results are comparable with HPLC results as shown in FIG. 13. The percentage errors are about 5% or less.

TABLE 1
Four standard samples measured in triplicates

| Standards | standard 1 | standard 2 | standard 3 | standard 4 |
|---|---|---|---|---|
| Loading (ug) | 50 | 117 | 183 | 250 |
| Peak area run1 | 420 | 1211 | 1756 | 2296 |
| Peak area run2 | 417 | 1162 | 1869 | 2564 |
| Peak area run3 | 470 | 1271 | 2091 | 2722 |
| Average peak area | 436 | 1215 | 1905 | 2527 |
| RSD (%) | 6.8 | 4.5 | 8.9 | 8.5 |

TABLE 2
Three real bioreactor samples measured in
triplicates and compared with offline HPLC results

| Samples | sample#1 | sample#2 | sample#3 |
|---|---|---|---|
| Peak area run1 | 1446 | 612 | 1008 |
| Peak area run2 | 1483 | 659 | 1001 |
| Peak area run3 | 1650 | 548 | 973 |
| Average peak area | 1526 | 606 | 994 |
| RSD (%) | 7.4 | 8.8 | 5.6 |
| Online Titer (mg/mL) | 0.300 | 0.125 | 0.205 |
| HPLC titer (mg/mL) | 0.306 | 0.128 | 0.224 |
| Percentage error (%) | 2.0 | 2.6 | 5.1 |

Although the invention has been described in terms of the preferred embodiments which constitute the best mode presently known for carrying out the invention, it should be understood that various changes and modifications as would be obvious to one having the ordinary skill in this art may be made without deviating from the scope of the invention which is defined by the claims appended hereto.

Various features of the invention are set forth in the claims that follow.

What is claimed is:

1. An antibody titer measurement device, comprising:
a housing having a motor controlled syringe connected with a buffer valve;
a temporary storage coil connected to the syringe and a sample valve;
a Protein A chromatographic column connected to the sample valve;
an ultraviolet detector; and
a waste liquid collector.

2. The device of claim 1, wherein the buffer valve is moved to select either neutral carrier buffer to enable binding of an antibody to a ProA chromatographic column or acidic buffer to elute the antibody.

3. The device of claim 1, wherein the sample valve having a plurality of external valve ports and a central connection port connecting to the temporary storage coil.

4. The device of claim 1, wherein the temporary storage coil acting as a temporary reservoir to enable delivery of an antibody sample to ProA column.

5. The device of claim 1, wherein the Protein A chromatographic column is to capture the antibody, to wash out impurities, and to release the antibody into the ultraviolet detector.

6. The device of claim 1, wherein the ultraviolet detector measures the ultraviolet signal of the eluted antibody.

7. A method of online measuring antibody titers in an antibody production, the method comprising:
moving a connection line on a buffer valve to a unit having neutral buffer, and filling a syringe with the neutral buffer;
moving the connection line on the buffer valve to a temporary storage coil, and moving the connection line of a sample valve to a Protein A chromatographic column, injecting the neutral buffer in the syringe into the temporary coil, flowing through the Protein A chromatographic column and an ultraviolet detector, and to a waste liquid collector;
moving the sample valve to a bioreactor, storing a certain volume of antibody sample from the bioreactor in the corresponding temporary storage coil;
moving the connection line in the buffer valve to neutral buffer, and filling the syringe with neutral buffer;
moving the connection line on the buffer valve to the temporary storage coil, and moving the connection line through the sample valve to the ProA chromatographic column, flowing the antibody sample in the temporary storage coil to the ProA column through the sample valve;
moving the connection line on the buffer valve to an acidic buffer, and filling the syringe with the acidic buffer; and
moving connection line on the motor to the temporary storage coil, the acidic buffer in the syringe flows into the temporary storage coil, through the sample valve to the ProA chromatographic column and to wash out the antibody sample into an ultraviolet detector.

8. The method of claim 7, further comprising collecting ultraviolet signals to calculate the concentration of antibody sample.
